(19) United States
(12) Patent Application Publication
(10) Pub. No.: US 2026/0325576 A1
(43) Pub. Date: Oct. 22, 2026

(54) COMPOSITION AND METHOD FOR TREATING MEIBOMIAN GLAND DYSFUNCTION

(71) Applicants: The University of Hong Kong, Pokfulam (CN); Advanced Biomedical Instrumentation Centre Limited, Hong Kong (CN)

(72) Inventors: Yau Kei Chan, Hong Kong (CN); Shuowen Zhou, Hong Kong (CN); Liangyu Zhou, Hong Kong (CN)

(21) Appl. No.: 19/646,854

(22) Filed: Apr. 14, 2026

Related U.S. Application Data
(60) Provisional application No. 63/790,848, filed on Apr. 18, 2025.

(51) Int. Cl. A61K 33/44; A61K 9/00; A61K 9/06; A61K 33/242; A61K 33/26; A61K 33/38; A61K 47/44; A61P 27/02

(52) U.S. Cl. CPC A61K 33/44; A61K 9/0048; A61K 9/06; A61K 33/242; A61K 33/26; A61K 33/38; A61K 47/44; A61P 27/02

(57) ABSTRACT

Disclosed herein is an ocular composition and method for treating Meibomian Gland Dysfunction (MGD) and related ocular conditions. The composition includes a light-sensitive microscale-dispersed eye ointment containing photosensitive particles, such as graphene, dispersed in a pharmaceutically acceptable carrier. Upon exposure to radiation such as near-infrared (NIR) radiation, the particles generate heat and/or reactive oxygen species (ROS), facilitating the melting of solidified meibum, relief of gland obstruction, reduction of ocular surface inflammation, and improvement in meibum quality. The formulation is administered topically to the eyelid, with delivery methods including ocular patches or gels. The disclosure provides a safe, effective, and non-invasive therapy for MGD and other ocular surface disorders.

### CROSS-REFERENCE TO RELATED APPLICATIONS

This application claims benefit of U.S. Provisional Application No. 63/790,848, filed Apr. 18, 2025, which is hereby incorporated herein by reference in its entirety.

### FIELD OF THE INVENTION

The disclosed invention is generally in the field of photosensitive particles for use in treating ocular conditions.

### BACKGROUND OF THE INVENTION

MGD is the leading cause of evaporative dry eye disease and one of the most common conditions encountered in ophthalmology clinics. The prevalence of MGD varies widely across published studies, but it is relatively high in Asian populations, ranging from 46.2% to 69.3%. Symptoms of MGD can significantly impact quality of life, leading not only to eye irritation but also to ocular surface inflammation and, ultimately, deficits in visual function. According to the International Workshop on Meibomian Gland Dysfunction (IWMGD), MGD is a chronic, diffuse abnormality of the meibomian glands (MG), commonly characterized by obstruction of the ducts with qualitative/quantitative changes in the glandular secretion.

It is an object of the present invention to provide composition and methods to treat Meibomian gland dysfunction (MGD) in an effective, economical, safe and user-friendly way.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

### BRIEF SUMMARY OF THE INVENTION

Formulations and methods for treating Meibomian gland dysfunction (MGD), are disclosed. The compositions include an effective amount of one or more photosensitive particles capable of generating heat and/or reactive oxygen species (ROS) upon exposure to radiation, in a pharmaceutically acceptable excipient, for administration to eye followed by effective irradiation to treat one or more symptoms of MGD.

The formulations are suitable for topical administration onto the eyelid. In some forms, the formulation is an ointment. In some forms, the formulation is a film or tape. In some forms, the formulation is a dosage form, shaped to conform to the size of the MGD site being treated for focused and targeted delivery of the graphene to the MGD site. The photosensitive particle can be a microparticle, a nanoparticle or a combination thereof for example, metal-based nanoparticles, non-metal based particles such as graphene, semiconductor materials or polymeric particles.

Methods of treating MGD in a subject in need thereof include administering the disclosed formulations to a site in need of treatment, the site being a site with MGD. The formulation is preferably applied to an eyelid of the subject, followed by application of an effective amount of irradiation, preferably Near infrared (NIR) light. The NIR can be provided by a NIR light source machine which includes a probe that can irradiate NIR laser beam; i.e., a portable probe, connected to a light source (with a picture), for example, an 808 nm light source can be used in the disclosed methods. The probe is aligned to the formulation, to deliver treatment. In some forms, the light source is a fixed instrument in a clinic. In some forms, the light source is a transportable device e.g., a handheld LED Infrared Light Therapy Pen®. In some forms, the distance for treatment is approximately 3-4 cm. In some forms, the duration of treatment is from about 5 minutes to about 60 minutes for 1 to 5 times daily for about 1 to 10 days for example, 2, 3, 4, 5, 6, 7, 8, 9 or 10 days, preferably, between about 3 days to about 5 days. In some forms, the duration of treatment is from about 10 min to 40 minutes for 1 to 3 times daily for 3 days. In some forms, treatment current is fixed at about 300 mA to reach an expected treatment temperature of around 41.3 °C.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

FIG. 1 is a schematic showing the concept of the NIR-responsive microparticles intreating MGD by applying heat to melt the solidified lipid in meibomian glands.

FIG. 2 shows solidified, granular meibum secretion observed upon meibomian gland compression.

FIG. 3 shows current-temperature curve of eyelid under NIR irradiation.

FIG. 4 shows evaluation of meibum quality after compression.

FIG. 5 shows intergroup comparisons of meibomian gland function scores (*p<0.05, **p<0.01, ***p<0.001, ****p<0.0001).

FIG. 6 shows intergroup comparisons of Schirmer I test results (*p<0.05, **p<0.01, ***p<0.001, ****p<0.0001).

FIG. 7 shows intergroup comparisons of corneal fluorescein staining scores (*p<0.05, **p<0.01, ***p<0.001, ****p<0.0001).

### DETAILED DESCRIPTION OF THE INVENTION

The disclosed method and compositions can be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### A. Definition

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied.

“Active Agent,” as used herein, refers to a physiologically or pharmacologically active substance that acts locally and/or systemically in the body. An active agent is a substance that is administered to a patient for the treatment (e.g., therapeutic agent), prevention (e.g., prophylactic agent), or diagnosis (e.g., diagnostic agent) of a disease or disorder. “Ophthalmic Drug” or “Ophthalmic Active Agent”, as used herein, refers to an agent that is administered to a patient to alleviate, delay onset of, or prevent one or more symptoms of a disease or disorder of the eye, or diagnostic agent useful for imaging or otherwise assessing the eye.

“Effective amount”, in reference to an active agent such as a self-assembling peptide or biomolecule, pharmaceutical agent, etc., refers to the amount necessary to elicit a desired biological response. As will be appreciated by those of ordinary skill in this art, the effective amount of an agent may vary depending on such factors as the desired biological endpoint, the agent to be delivered, the nature of the site to which the agent is delivered, the nature of the conditions for which the agent is administered, etc. For example, the effective amount of a composition for treatment of a disease or disorder may be an amount sufficient to promote recovery to a greater extent than would occur in the absence of the composition.

“Preventing” refers to causing a condition, state, disease, symptom or manifestation of such, or worsening of the severity of such. Preventing includes reducing the risk that a condition, state, or disease, or symptom or manifestation of such, or worsening of the severity of such, will occur.

The terms “treat”, “treatment” and “treating” refer to the reduction or amelioration of the progression, severity and/or duration of one or more symptoms of an injury, disease or disorder, delay of the onset of a disease or disorder, or the amelioration of one or more consequences, indications or symptoms (preferably, one or more discernible symptoms) of an injury, disease or disorder, resulting from the administration of one or more therapies (e.g., one or more therapeutic agents such as a compound as described). The terms “treat”, “treatment”, and “treating” also encompass the reduction of the risk of developing a disease or disorder, and the delay or inhibition of the recurrence of a disease or disorder.

The terms “increase,” “enhance,” “stimulate,” and/or “induce” (and like terms) generally refer to the act of improving or increasing, either directly or indirectly, a function or behavior relative to the natural, expected, or average or relative to current conditions. For instance, something that increases, stimulates, induces or enhances anti-inflammatory effects might induce the production, and/or secretion of anti-inflammatory cytokines, and/or infiltration of immune cells that mediate anti-inflammatory responses, such as Treg, or Th17 cells.

The term “subject” or “individual” includes both humans, mammals (e.g., cats, dogs, horses, etc.), and other living species that are in need of treatment for inflammatory and/or autoimmune conditions/diseases.

The term “carrier” or “excipient” refers to an organic or inorganic, natural or synthetic inactive ingredient in a formulation, with which one or more active ingredients are combined. In some embodiments, a carrier or an excipient is an inert substance added to a pharmaceutical composition to further facilitate administration of a compound, and/or does not cause significant irritation to an organism and does not abrogate the biological activity and properties of the administered compound.

### B. Composition

Disclosed herein is an ocular composition that includes one or more photosensitive particles capable of generating heat and/or reactive oxygen species (ROS) upon exposure to radiation; and optionally, a pharmaceutically acceptable ocular carrier wherein the ocular composition is suitable for application to eyelids.

### 1. Photosensitive Particles

Disclosed herein is a composition that includes photosensitive particles such as but are not limited to a microparticle, a nanoparticle or a combination thereof.

In some forms, the photosensitive particles are microparticles. In some forms, the photosensitive particle is made from graphene. In some forms, the microparticles are made from graphene related materials (such as Graphene oxide, carbon micro-tubes, polydopamine coated carbon micro-particles, carbon micro-gels); metal and metal oxides materials (such as copper sulfide microparticles, iron oxide microparticles); Polypyrrole nanogel; transition metal dichalcogendes (such as molybdenum disulfide MoS2, tungsten diselenide WSe2). In some forms, the photosensitive particles include but are not limited to metal-based nanoparticles such as gold nanoparticles, silver nanoparticles, iron oxide nanoparticles; carbon-based nanostructures such as carbon nanotubes, graphene derivatives, and fullerenes; semiconductor materials such as titanium dioxide, metal oxide semiconductors, quantum dots; organic and polymeric materials such as indocyanine green, porphyrin derivatives, polymeric nanoparticles, and light-sensitive microspheres and microcapsules.

Graphene is a carbon allotrope and is a single layer of carbon atoms arranged in a hexagonal honeycomb lattice, making it a two-dimensional material with exceptional properties like high electrical conductivity, incredible strength, flexibility, and being extremely lightweight. Graphene is derived from graphite, where multiple layers of graphene are stacked together. In some forms, the graphene is in a form of a micro-size sheet. In some forms, the graphene is in a form of a fullerene.

In some forms, the photosensitive particles in the composition have sizes of about 1 nm-1000 μm, 10 nm-500 μm, 50 nm-250 μm, 100 nm-150 μm, 200 nm-100 μm, 500 nm-50 μm, or 1 μm-25 μm.

In some forms, the photosensitive particles in the composition are present in a concentration of about 0.001 μg/mg-5.0 μg/mg, 0.005 μg/mg-2.5 μg/mg, 0.01 μg/mg-1.0 μg/mg, 0.015 μg/mg-0.95 μg/mg, 0.02 μg/mg-0.9 μg/mg, 0.03 μg/mg-0.8 μg/mg, 0.04 μg/mg-0.7 μg/mg, 0.05 μg/mg-0.6 μg/mg, 0.06 μg/mg-0.5 μg/mg, 0.07 μg/mg-0.4 μg/mg, 0.08 μg/mg-0.3 μg/mg, 0.09 μg/mg-0.2 μg/mg. In some forms, the photosensitive particles are present in a concentration of 0.1 μg/mg.

Photosensitive particles disclosed herein facilitate the generation of heat and/or reactive oxygen species (ROS) upon exposure to radiation, particularly near-infrared (NIR) light. The photothermal response of these particles facilitate melting of solidified meibum, alleviating meibomian gland obstruction and improving tear film stability. In some forms, these particles are dispersed in a pharmaceutically acceptable carrier to facilitate uniform distribution and sustained retention at the treatment site.

In some forms, the photosensitive particles disclosed herein facilitate the generation of heat and/or reactive oxygen species (ROS) upon exposure to radiation is a photoactive heterojunction material.

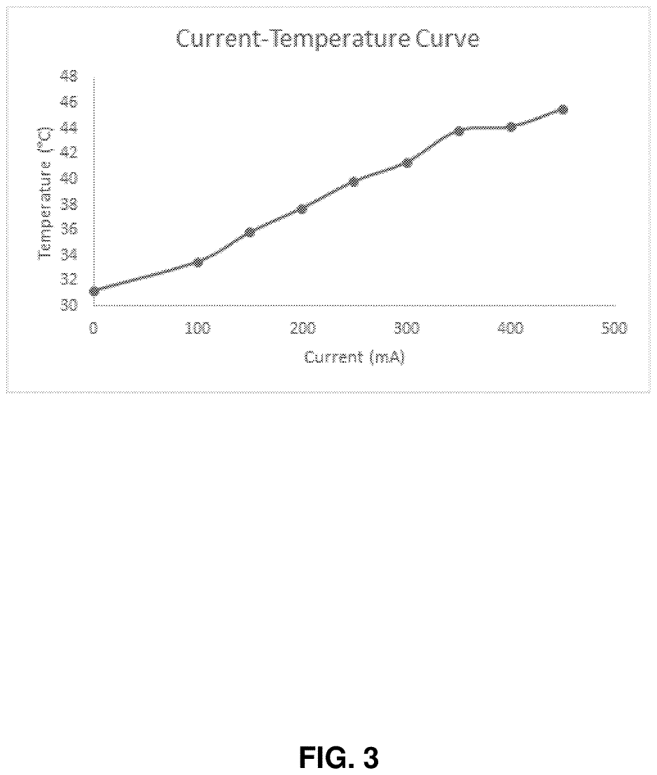

Photoactive heterojunction materials are known in the art and include, but are not limited to materials containing titanium dioxide (TiO2), copper (Cu2), and Zinc oxide (ZnO). Heterojunction photocatalysts/photoactive heterojunction materials and methods of making the same, are known in the art (reviewed in Mutalik et al. Nanomaterials, Vol. 10(6):643 (2020), doi:10.3390/nano10040643 and Zhou et al., ACS Appl. Bio. Mater., Vol. 4, pages 3909-3936 (2021) and Low, et al., (2017). Heterojunction photocatalysts. Advanced materials, 29(20), 1601694.). A heterojunction, in general, is defined as the interface between two different semiconductors with unequal band structure, which can result in band alignments. Typically, there are three types of conventional heterojunction photocatalysts, those with a straddling gap (type-I), those with a staggered gap (type-II), and those with a broken gap (type-III) (see FIG. 3 in Low, et al., (2017). Heterojunction photocatalysts. Advanced materials, 29(20), 1601694.).

Exemplary combinations of photoactive heterojunction materials include but are not limited to Graphene oxide/copper ferrite nanoparticles (Go/CFN), Silver-titanium oxide (Ag—TiO2), TiO2/AgVO3, Ag/TiO2/cellulose, Alginate/Au—TiO2, Lithium-titanate in the low-density polyethylene matrix (Li—TiO2/LDPE), Cu—TiO2, TiO2/—Fe2O3, TiO2 doped with Bi (Bi—TiO2 group) and TiO2 co-doped with urea and Bi (Urea, Bi—TiO2 group; U,Bi—TiO2), chitosan films containing melon/TiO2 (CTS/MTiO2), TiO2 nanoparticles and graphene sheets (TiO2/GSs), graphene oxide and cuprous oxide (rGO-Cu2O), and Zinc oxide-selenium (ZnO—Se). The amount of photoactive materials that can be incorporated into the hydrogel range from about 50 μg to about 250 pg, preferably from about 80□g to about 200 □g.

In preferred embodiments, the photoactive heterojunction material is formed of graphene oxide (Go) and Copper ferrite nanoparticles (CFN). Bio-heterojunction materials of copper ferrite nanoparticle (CFN) and graphene oxide (GO) can destroy pathogenic bacteria through synergistic strategy of PDT and PTT. Accompanied by multivalent copper (Cu) and iron (Fe), copper ferrite nanoparticles (CFN, CuFe2O4), possess excellent NIR absorbance for photothermal effect and Fenton(-like) reaction activity. GO as a class of two-dimensional biocompatible material, provides binding sites to CFN for formation of hetero-interface. After hybridization into GO/CFN bio-heterojunction, the hetero-interface between GO and CFN can produce singlet oxygen (1O2) under NIR exposure to eradicate pathogenic bacteria through PDT. Meanwhile, the released copper ions can improve corneal re-epithelialization and promote the process of wound healing.

Methods of making photoactive heterojunction materials are known. For example, heterojunction material formed of graphene oxide (Go) and Copper ferrite nanoparticles (CFN) (Go/CFN) can be synthesized via a hydrothermal reaction. As described in the non-limiting Examples, a desired amount of Go nanosheets are dissolved ethylene glycol. Then, an amount of iron chloride hexahydrate, copper chloride dihydrate, sodium acetate, and polyvinylpyrrolidone are added to the Go-ethylene glycol solution. The mixture is then ultrasonicated and transferred to a Teflon autoclave. The hydrothermal reaction is kept at a suitable temperature for a pre-determined amount of time e.g., 180 °C for 24 hours. Finally, the Go/CFN heterojunction powders are collected, rinsed using ethyl-alcohol and deionized (D.I.) water and dried.

The bioactivity produced from the inclusion of photoactive heterojunction materials in the antimicrobial hydrogel composition depends on the principle of photocatalysis.

Photocatalysis is mainly a process in which the electrons generated from photosensitizer (PS) are excited by light, an excited state with the subsequent redox reactions with the reactants (Reviewed in Zhou et al., ACS Appl. Bio. Mater., Vol. 4, pages 3909-3936 (2021)). The photocatalytic antibacterial process is the reaction between bacteria and ROS. As an intermediate product, ROS mainly results from an incomplete reduction reaction between light-excited electrons/holes and oxygen-(O₂) or water during the photocatalytic reaction process, which exhibits higher activity (Jia, et al. Rejuvenated Photodynamic Therapy for Bacterial Infections. Adv. Healthcare Mater. 2019, 8, 1900608; Wang, Y. et al. Construction of nanomaterials with targeting phototherapy properties to inhibit resistant bacteria and biofilm infections. Chem. Eng. J. 2019, 358, 74-90). ROS mainly include singlet oxygen (¹O₂), superoxide anions (·O₂—), hydroxyl radicals (·OH), etc. (Wang, L et al. Using porous magnetic iron oxide nanomaterials as a facile photoporation nanoplatform for macromolecular delivery. J. Mater. Chem. B 2018, 6, 4427-4436). For example, in an embodiment incorporating the metal oxide TiO₂, when the energy of light is higher than the band gap of TiO₂, the electrons can jump from a valence band (VB) to a conduction band (CB), forming electron-hole pairs (Mao, et al. Repeatable photodynamic therapy with triggered signaling pathways of fibroblast cell proliferation and differentiation to promote bacteria-accompanied wound healing. ACS Nano 2018, 12, 1747-1759). Holes can interact with OH— or H₂O to generate ·OH. The ·OH is a highly active species that can oxidize a variety of organic substances indiscriminately. Photogenerated electrons can also interact with O₂ to generate other ROS such as ·O₂—, and these reactive oxygen radicals can also participate in redox reactions. The reaction process is shown in the following equations:

TiO₂+hv→TiO₂(e⁻,h⁺) (1)

e⁻+h⁺→heat or hv (2)

$${h}^{+} + {OH}_{ads}^{-} \to {HO}^{*} \qquad ( 3 )$$

h⁺+H₂O_{ads}→HO·+H⁺ (4)

e⁻+O₂→O₂⁻· (5)

Thus, photocatalytic therapy depends a photosensitive reaction accompanied by subsequent biological effects. This process is that a specific wavelength of laser radiation excites a photosensitizer (PS) at the bacterial infected sites, and the produced photogenerated electrons or holes react with surrounding oxygen or water to generate highly active oxygen radicals (Sun, J. et al., Synergistic photodynamic and photothermal antibacterial nanocomposite membrane triggered by single NIR light source. ACS Appl. Mater. Interfaces 2019, 11, 26581-26589).

### 2. Carrier

In some forms, the disclosed composition also contains a carrier. Any suitable carrier for delivering lipophilic material can be used. In some forms, the carrier is oil based.

In some forms, lipophilic material or oil-based materials include lanolin and/or petroleum jelly. In some forms lipophilic material or oil-based materials include but not limited to mineral oil, vegetable oil, wax, silicone oil, castor oil, olive oil, coconut oil, jojoba oil, shea butter, cocoa butter, or synthetic esters.

In some forms, the carrier includes liposomes, liposome-like nanoparticles, solid lipid nanoparticles, nanostructured lipid carriers, absorbed lipid-polymer hybrid nanoparticles, encapsulated lipid-polymer hybrid nanoparticles, exosomes, native lipoprotein, and synthetic lipoprotein.

The choice of a pharmaceutically acceptable ocular carrier is important for ensuring effective delivery, stability, and patient comfort. The carrier serves as the medium in which the active photosensitive particles, such as graphene, are dispersed, facilitating their application to the eyelids and subsequent therapeutic action upon irradiation.

A suitable ocular carrier should be non-toxic and well-tolerated by ocular tissues to prevent irritation or adverse reactions. It should maintain the stability of the active ingredient, preventing degradation or aggregation over time. The carrier's viscosity should allow for easy application while ensuring adequate retention time on the ocular surface. For photothermal therapies, the carrier should not obstruct or absorb therapeutic light wavelengths, ensuring efficient activation of the photosensitive particles.

In some forms, the carrier material is a hydrogel configured for delivery of lipophilic active agents. Hydrogels can be used as lipophilic carriers by incorporating lipid nanoparticles (LNPs) or nanostructured lipid carriers (NLCs) into the hydrogel matrix, allowing for controlled and enhanced delivery of lipophilic drugs. LNPs and NLCs are lipid-based nanoparticles that can effectively encapsulate lipophilic drugs, improving their solubility and stability. By embedding LNPs/NLCs within a hydrogel matrix, a dual-delivery system is created. The hydrogel provides a biocompatible and tunable matrix for controlled drug release, while the LNPs/NLCs enhance the encapsulation and delivery of the lipophilic drug. Various hydrogel polymers, such as alginate and chitosan, and lipid nanoparticles, such as NLCs and liposomes, are used in these composite systems.

Hydrogels are three-dimensional (3D) cross-linked polymer networks, which can absorb and retain large amount of water. A hydrogel is a three-dimensional (3D) network of hydrophilic polymers that can swell in water and hold a large amount of water while maintaining the structure due to chemical or physical cross-linking of individual polymer chains. Hydrogels are mainly formed from biopolymers and/or polyelectrolytes. Hydrogels useful in the disclosed composition can be made using any polymeric material that is suitable for ocular applications

Such materials used for the preparation of hydrogels are known in the art and include for example, polymers with quaternary ammonium functional groups, natural peptides such as collagen, gelatine, polysaccharides such as starch, alginate, and agarose, phospho- and sulfo-derived polymers, phenol and benzoic acid derived polymers, and polyelectrolyte multilayers (Ahmed, J Adv Res. 2015 March; 6(2): 105-121; Pierau and Versace, Materials (Basel), 14(4): 787 (2021)). Other examples include covalently crosslinked hyaluronic acid, and the hyaluronic acid may be modified or unmodified. Unmodified hyaluronic acid may be covalently crosslinked by a variety of methods, including crosslinking using 1,4-butanediol diglycidyl ether (BDDE), divinylsulfone, and dihydrazide. The hyaluronic acid may be modified to change the charge of the molecule, change its biological activity, or to include groups that may be used for crosslinking purposes. Particularly useful are thiolated hyaluronic acid or thiolated carboxymethyl hyaluronic acid. Modified hyaluronic acid may be crosslinked with an external molecule for crosslinking, or without an external crosslinker molecule. For crosslinking thiolated HA or CMHA, a molecule with thiol-reactive sites, such as acrylates, methacrylates, haloacetates, haloacetamides, or maleimides, may be used as an external crosslinker molecule, examples of which include poly(ethylene glycol) diacrylate and poly(ethylene glycol) bisbromoacetate. For crosslinking without an external crosslinker molecule, in particular, thiolated HA or CMHA may be disulfide crosslinked via an oxidation process. Such disulfide crosslinking may be aided by use of an oxidant such as sodium hypochlorite or peroxide.

When modified HA is crosslinked via the modification (e.g., disulfide crosslinking of thiolated HA), the level of modification may be adjusted to control the amount of crosslinking of the hydrogel, such that a higher level of modification leads to more crosslinking. Particularly useful for formulating hydrogels of the present disclosure is thiolated HA or thiolated CMHA, where the thiol modification is about 0.05 to about 1.0 mol thiol per mg of HA or CMHA. Modification levels within this range are particularly suitable for forming crosslinked hydrogels with a desired shear-thinning profile and viscosity.

In a preferred embodiment, the polymer used to form the hydrogel base is poly-epsilon-lysine (PεK), resulting in a hydrogel with intrinsic antibacterial activity. Poly-epsilon-lysine (pεK) is a cationic peptide with intrinsic antimicrobial properties and broad-spectrum antimicrobial activity against gram-positive and -negative bacteria, yeasts, and fungi. ε-polylysine is a kind of natural polymeric material forming hydrogel, it is obtained by microbial fermentation. The molecular weight of ε-polylysine is between 3 kDa and 5 kDa. A ε-polylysine hydrogel, it has the following constitutional unit:
wherein, n is any natural number of 20 to 30, m is any natural number of 50 to 70. Epsilon-polylysine is available from BOC Sciences, Catalog NO.: B0001-011213 CAS NO.: 28211-04-3. Methods for making PεK hydrogels, are known in the art (Aveyard et al. ACS Appl Mater Interfaces 2019; 11(41):37491-50).

The hydrogel can be one with intrinsic antibacterial activity such as Fmoc-diphenylalanine and Fmoc-capped short peptides bearing either lysine-rich or pyridinium groups (have been found with moderate antibacterial activities against Gram-positive and Gram-negative bacteria); anthranilamide-based diphenylalanine peptide mimics formed self-assembled hydrogels; hydrogels prepared by self-assembly of naphthyl anthranilamide (NaA) capped amino acid based cationic peptide mimics (Reviewed in Aldilla, et al., Scientific Reports volume 12, Article number: 22259 (2022)).

For example, McCloskey et al discloses Fmoc-peptides (FmocFF (a), FmocFFKK (b), Fmoc-FFFKK (c) and FmoCFFOO (d), shown below) which formed surfactant-like soft gels at concentrations of 1% w/v.

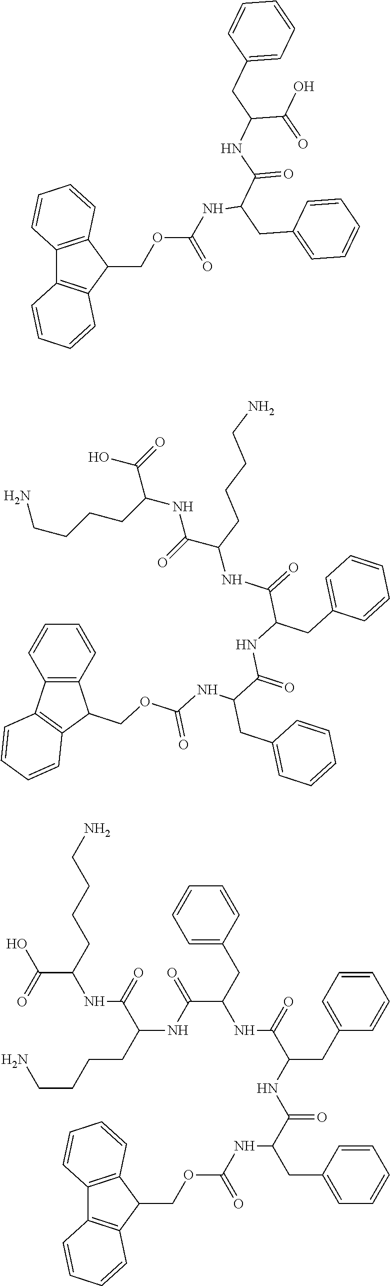 [SMILES: O=C(NC(Cc1ccccc1)C(=O)NC(Cc1ccccc1)C(=O)O)OCC1c2ccccc2-c2ccccc21 ; NCCCCC(NC(=O)C(CCCCN)NC(=O)C(Cc1ccccc1)NC(=O)C(Cc1ccccc1)NC(=O)OCC1c2ccccc2-c2ccccc21)C(=O)O ; NCCCCC(NC(=O)C(CCCCN)NC(=O)C(Cc1ccccc1)NC(=O)C(Cc1ccccc1)NC(=O)C(Cc1ccccc1)NC(=O)OCC1c2ccccc2-c2ccccc21)C(=O)O]

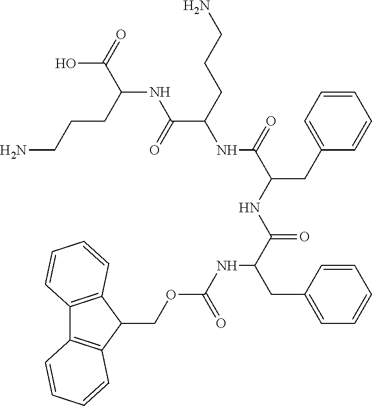 [SMILES: NCCCC(NC(=O)C(CCCN)NC(=O)C(Cc1ccccc1)NC(=O)C(Cc1ccccc1)NC(=O)OCC1c2ccccc2-c2ccccc21)C(=O)O]

(McCloskey, A. P., Draper, E. R., Gilmore, B. F. & Laverty, G. Ultrashort self-assembling Fmoc-peptide gelators for anti-infective biomaterial applications. J. Pept. Sci. 23, 131-140 (2017)). Other exemplary peptides are disclosed in Debnath, et al., Phys. Chem. B 2010, 114, 13, 4407-4415.

Liposomes are artificial “fat bubbles”, i.e., vesicles, characterized by having one or more bilayers spontaneously achieved by dispersal of natural or synthetic amphipathic lipids in water. Liposomes are spherical vesicles comprising unilamellar or multilamellar phospholipid bilayers enclosing an aqueous core in which the drug of choice can be encapsulated. They are prepared from materials possessing polar head (hydrophilic)groups and nonpolar tail (hydrophobic) groups. The interaction between these groups induces the formation of vesicles. Pharmaceutics 13, 206 (2021) https://doi.org/10.3390/pharmaceutics13020206.

Nanostructured Lipid Carriers (NLC) are nano-sized colloidal drug delivery system that contains a lipid mixture consisting of both solid and liquid lipids in their core. This Lipid-Based Nanosystem is introduced as a biocompatible, non-toxic, and safe nano-drug delivery system as compared to polymeric or metallic nanoparticles. NLCs as the name suggest are nanosized multiparticulate system in the size range of 50 nm to 500 nm. The particle size distribution of NLC depends on nanoparticles' manufacturing process and composition. Colloidal in nature this particle resembles the structure of SLN with the main variation being in the core of these nanoparticles. Unlike SLN which contains a solid lipid core in which lipid is arranged in a highly organized fashion, NLC contains lipid liquid along with a solid lipid core which forms an unorganized drug matrix. With this unorganized nature, more drugs can be loaded in the core in addition to stability issues encountered on long-term storage of SLN due to crystallization and drug expulsion is also rectified with NLC (reviewed in Khan, et al., Adv Pharm Bull. 2022 Sep. 18; 13(3):446-460).

Lipid-polymer hybrid nanoparticles (LPHNPs) are next-generation core-shell nanostructures, conceptually derived from both liposome and polymeric nanoparticles (NPs), where a polymer core remains enveloped by a lipid layer. As can be inferred from their name, LPHNPs merge the features of both polymeric NPs and liposomes. They consist of three building blocks: 1) a polymer core encapsulating the drug, 2) a lipid mono-layer surrounding the polymer core, and 3) an outer lipid-PEG layer, a steric stabilizer prolonging systemic circulation of the LPHNPs by evading immune destruction. The middle lipid monolayer behaves like a molecular barricade that mitigates the loss of entrapped drugs over the course of the LPHNP formulation and protects the core from degradation by preventing the diffusion of water into the inner core. LPHNP and methods of making thereof, are reviewed in Mukherjee, et al., Int J Nanomedicine. 19; 14:1937-1952.

### 3. Combination Therapy

In some forms, the ocular composition disclosed herein can be combined with one or more anti-inflammatory agents.

In some forms, the ocular composition can be combined with one or more of corticosteroids, cyclosporine, azithromycin.

Topical corticosteroids, such as dexamethasone, are effective in reducing ocular surface inflammation. Short-term use can alleviate symptoms. Cyclosporine is an immunomodulatory agent that decreases inflammation by inhibiting T-cell activation. Formulations like 0.05% cyclosporine have been utilized to improve tear production and reduce inflammatory markers in dry eye conditions. Beyond its antimicrobial properties, azithromycin exhibits anti-inflammatory effects by reducing pro-inflammatory cytokines and matrix metalloproteinases. Topical application has shown efficacy in improving meibomian gland function and alleviating associated symptoms.

Combining the disclosed ocular composition with anti-inflammatory agents is capable of enhancing therapeutic outcomes for conditions such as Meibomian Gland Dysfunction (MGD) by addressing both gland obstruction and ocular surface inflammation. This dual approach facilitates the clearance of meibomian gland blockages and mitigates the inflammatory processes contributing to ocular conditions such as MGD pathology.

### 4. Pharmaceutical Formulation

The disclosed composition can be formulated into various dosage forms suitable for ocular application or application in the eyelids, including, but not limited to, an ophthalmic gel, an ocular patch, a dissolvable or bioadhesive ocular film, an ocular wound bandage, or a lipid-based ointment. In some forms, the formulation may be adapted into a slow-release form capable of gradual diffusion of the active ingredient over an extended period.

### Ophthalmic Gels

In some forms, disclosed composition is in the form of an ophthalmic gel. Ophthalmic gels are semi-solid systems that offer prolonged contact with the ocular surface, enhancing drug bioavailability. Their viscosity reduces the drainage rate, allowing sustained release of the active ingredient. In some forms, polymers such as chitosan and xyloglucan are used to formulate these gels due to their biocompatibility and mucoadhesive properties.

### Ocular Patches

In some forms, disclosed composition is in the form of an ocular patch. Ocular patches are designed to adhere to the ocular surface or eyelids, providing a controlled and sustained release of therapeutic agents. These patches can be fabricated using biodegradable polymers that ensure prolonged retention and gradual drug release. The design of ocular patches considers factors such as permeability, adhesiveness, and patient comfort to ensure efficacy and compliance.

### Dissolvable or Bioadhesive Ocular Films

In some forms, disclosed composition is in the form of a dissolvable or bioadhesive ocular film. These thin films are placed on the ocular surface, where they dissolve or adhere to release the active ingredient over time. Materials like hydroxypropyl pea starch can be utilized to create films for ocular administration, offering potential as carriers for drugs like azelastine hydrochloride. Such films provide a sustained release profile and are designed to minimize discomfort and visual disturbance.

### Ocular Wound Bandages

In some forms, disclosed composition is in the form of an ocular wound bandage. Ocular wound bandages serve as protective coverings that can deliver therapeutic agents while promoting healing. Incorporating the ocular composition into a biodegradable bandage can provide localized therapy, maintaining the necessary concentration of the active ingredient at the site of action. Materials such as polyethylene glycol (PEG)-based hydrogels have been explored for their adhesive properties and ability to form stable, biocompatible barriers. Medicated ocular bandages are reviewed in (Zidan, et al., Pharm Dev Technol, 23(3):255-260 (2017).

### Lipid-Based Ointments

In some forms, disclosed composition is in the form of a lipid-based ointment. Lipid-based ointments provide prolonged retention on the ocular surface due to their viscous nature, enhancing drug bioavailability. A common choice for ointment base is white petrolatum, known for its appropriate melting point, resulting in reduced viscosity post-administration.

### Aerosolized or Mist Formulation

In some forms, the composition may be adapted into an aerosol spray or fine mist for enhanced distribution over the ocular surface, for even absorption and maximum therapeutic efficacy.

### C. Methods of Use

Methods for treating ocular disease, ocular disorder or ocular condition in a subject are provided. The disclosed method includes applying a therapeutically effective amount of the disclosed compositions to a subject in need thereof. The method includes contacting the eye of a subject in need thereof, with an effective amount of the disclosed composition.

The method includes contacting the composition in the subject's eye with radiation having wavelength anywhere between 380-1200 nm. In some forms, the radiation has a wavelength of about 380 nm-1200 nm, 400 nm-1000 nm, 500 nm-900 nm, 550 nm-800 nm, 600 nm-750 nm, 1000 nm-1200 nm, 800 nm-1100 nm, or 900 nm-1000 nm. In some forms, the method includes contacting the composition in the subject's eye with near infrared radiation (NIR). In some forms, the radiation is released from a portable handheld device such as handheld LED. In some forms, a source of the radiation includes one or more of a handheld device, a stationary light delivery system, a wearable device, a light-emitting diode (LED) array, a laser system, a broadband light source, a filtered light source, an intense pulsed light (IPL) device, or any combination thereof.

In some forms, the radiation source is a fixed instrument in a clinic. In some forms, the duration of treatment is from about 1 min-20 min, 3 min-17 min, 5 min-15 min, 7 min-13 min, 8 min-12 min, 9 min-11 min, or 9-10 min for 1 day, 1-2 days, 1-3 days, 1-4 days or 1-5 days, 1-6 days, or 1-7 days.

In some forms, the light exposure induces heat generation at the target site. In some forms, the treatment temperature is maintained for 38 °C, 39 °C, 40 °C, 41 °C, 42 °C, 43 °C, 44 °C, 45 °C, or 46 °C, preferably 41.3 °C, which is sufficient to melt solidified meibum without causing damage to the eyelid or cornea.

The therapeutic effects disclosed herein are attributed to the photothermal response of the photosensitive particles upon radiation exposure, facilitating multiple key mechanisms. The localized heat melts solidified meibum, restoring normal glandular function, while the increased temperature promotes glandular secretion, alleviating meibomian gland obstruction. Additionally, the radiation generates reactive oxygen species (ROS) and controlled hyperthermia help reduce ocular surface inflammation by mitigating inflammatory mediators associated with MGD. The photothermal process also improves meibum quality by enhancing its consistency and flow, which is essential for tear film stability. Furthermore, the heat and ROS production contribute to microbial control, reducing bacterial overgrowth commonly linked to MGD, thereby ensuring a comprehensive and effective treatment approach.

In some forms, the treatment described herein using the disclosed composition improves meibomian gland function, as depicted by reduced Meibomian Gland Function Scoring compared to control groups in the Example section. In some forms, enhanced tear secretion was observed in the Schirmer I test, indicating improved ocular surface lubrication. The corneal fluorescein staining score indicated a notable reduction in corneal epithelial defects, indicating effective restoration of ocular surface integrity. No notable adverse effects, including burns or ocular irritation, were observed following treatment.

In some forms, table 1 provides known treatments for MGD.

TABLE 1
The comparative analysis of known treatments for MGD

| | | Temporary | | | | | | | |
|---|---|---|---|---|---|---|---|---|---|
| | | relief of dry eye | | | | | | | |
| | | symptoms (R)/ | | | | | | | |
| | | Fundamental | | | | | | | |
| | | solution for | | | | | | Cost | |
| Treatment | Mechanism of action | MGD (S) | Treatment frequency | Anaesthesia | Efficacy | Adverse effects | Availability | Machine* | Per treatment* |

Conventional Physical Therapies

| | | | | | | | | | |
|---|---|---|---|---|---|---|---|---|---|
| Warm | Reduces meibum | S | Once to twice | Not required | Shows short-term improvements | Low incidence of adverse | Easily | N/A | $ |
| compresses (e.g. | viscosity and | | a day when | | in ocular symptoms, TBUT, and | events (AEs) | accessible, | | |
| I-RELIEF ®, | lysophospholipid | | symptoms arise | | lipid layer thickness of the tear | | can be done in | | |
| EyeGiene ®, | levels using direct | | | | film | | home setting. | | |
| MGDRx eye | wet or dry heat | | | | | | | | |
| bags ®) | applied to eyelid | | | | | | | | |
| Eyelid cleansing | Reduces MG | S | Once a day, | Not required | Significantly improvements in | Transient eye irritation | Easily | N/A | $-$$ |
| (e.g. | obstruction by | | for longer | | tear film lipid layer and reduced | and burning sensations | accessible, | | |
| OCuSOFT ®, | removing debris and | | than 6 months | | ocular inflammation | | can be done in | | |
| TheraTears ®) | dead skin cells from | | depending on | | | | home setting | | |
| | the eyelid | | age, ocular | | | | | | |
| | | | surface | | | | | | |
| | | | inflammation | | | | | | |
| | | | and MGD | | | | | | |
| | | | subtype | | | | | | |
| Probing and | Unplugs stagnant | S | Multiple | Anaesthetic | Immediate relief post treatment | Mild pain, transient | Office | $$$ | $$ |
| meibomian | meibum to promote | | sessions | eye drops or | with restored meibum levels and | conjunctivitis and | procedure | | |
| gland | normal gland | | recommended | ointments | significantly improved viscosity, | puffiness. Low incidence | requiring | | |
| expression | secretion and flow | | for long term | are often | but needs to be done regularly to | of lid haemorrhages | specialised | | |
| (MGX) | | | symptomatic | administered | maintain effect long term | | tools and | | |
| | | | relief | | | | expertise | | |

Pharmaceutical interventions

| | | | | | | | | | |
|---|---|---|---|---|---|---|---|---|---|
| Antibiotics (e.g. | Inhibits growth of | S | Azithromycin | Not required | Short term improvements in | Prevalent adverse events | Requires | N/A | $-$$ |
| azithromycin, | lipase-active | | is commonly | | clinical signs, with azithromycin | (AEs); oral administration | prescription | | |
| doxycycline, | commensal bacteria | | prescribed for | | being more effective than | associated with GI upset, | | | |
| minocycline, | that otherwise | | five days | | doxycycline | nausea, photosensitivity; | | | |
| sul facetamide) | destabilise the tear | | while | | Improvements usually reverted | topical eye drops | | | |
| | film | | doxycycline | | upon drug cessation | associated with ocular | | | |
| | | | is given for | | | irritation, hypersensitivity | | | |
| | | | one month | | | | | | |
| Anti- | Interferes with the | S | Corticosteroid | Not required | Low-dose corticosteroids | No major AEs | Opthalmic | N/A | $ |
| inflammatory | underlying | | ointment: | | improve Schirmer scores and | | lifitegrast and | | |
| agents (e.g. | inflammatory | | three or four | | decrease OSDI and SANDE | | corticosteroids | | |
| prednisolone, | pathways in MGD | | times a day; | | indices; Lifitegrast improves eye | | requires | | |
| betamethasone, | conditions | | Lifitegrast: | | dryness compared to placebo | | prescription | | |
| loteprednol, | | | one drop, | | | | | | |
| lifitegrast) | | | twice a day, | | | | | | |
| | | | administered | | | | | | |
| | | | regularly | | | | | | |
| | | | rather than as | | | | | | |
| | | | needed | | | | | | |
| Oral fatty acid | Increases levels of | S | Four times | Not required | Mixed efficacy; generally report | GI upset that resolved | Omega-3 fatty | N/A | $ |
| supplements | unsaturated fatty | | per day | | improvements in clinical signs | after stopping medication | acids available | | |
| (e.g. omega-3 | acids in meibum | | | | | | commercially | | |
| fatty acid) | secretions, | | | | | | | | |
| | promoting better | | | | | | | | |
| | flow; also shows | | | | | | | | |
| | anti-inflammatory | | | | | | | | |
| | properties | | | | | | | | |
| Diquafosol | Binds to P2Y2 | S | Six times per | Not required | Significant increase in lipid layer | No major AEs | Currently | N/A | $ |
| | receptor as an | | day | | thickness with reduced number of | | available and | | |
| | agonist, stimulating | | | | telangiectasias and plugged gland | | authorized in | | |
| | mucin secretion | | | | orifices | | Japan and | | |
| | from conjunctival | | | | | | other Asian | | |
| | goblet cell | | | | | | countries | | |
| Maxacalcitol | Acts as vitamin D3 | S | Twice per day | Not required | Reduces plugging of MG orifices | No major AEs | Requires | N/A | Unclear |
| | analogue to | | for 8 weeks | | and increases meibomian gland | | prescription | | |
| | suppress | | | | area | | | | |
| | keratinocyte | | | | | | | | |
| | proliferation, | | | | | | | | |
| | reducing MG | | | | | | | | |
| | obstruction | | | | | | | | |
| N-acetylcysteine | Acts as antioxidant | S | Four times | Not required | Significant improvements in | No major AEs | Available | N/A | $ |
| | to neutralise ROS in | | per day for 1 | | clinical symptoms; similar | | OTC, can be | | |
| | ocular tissues, | | month | | efficacy to topical steroid- | | done in home | | |
| | reducing oxidative | | | | antibiotic combination | | setting | | |
| | stress in MGD | | | | | | | | |
| Manuka honey | Reduces oxidative | S | Up to three | Not required | Showed significant | No major AEs | Available | N/A | $ |
| (e.g. Optimel | stress due to the | | times a day | | improvements in meibum quality | | OTC, can be | | |
| Manuka + Dry | antimicrobial and | | | | and gland expressibility with | | done in home | | |
| Eye Drops) | wound-healing | | | | reduced total eyelid marginal | | setting | | |
| | properties of | | | | bacterial colony counts | | | | |
| | Manuka honey gel | | | | | | | | |
| Artificial Tears | Supplement and | R | Depends on | Not required | Temporary relief of buming and | Temporary blurred vision, | Available | N/A | $ |
| (e.g. REFRESH | stabilize the tear | | the type, | | irritation due to dryness of the eye | minor eye | OTC, can be | | |
| TEARS ® | film | | usually | | | burning/stinging/irritation, | done in home | | |
| ALCON | | | several times | | | eye redness | setting | | |
| Systane ®, | | | per day | | | | | | |
| VISINE ® | | | | | | | | | |

Other treatment modalities

| | | | | | | | | | |
|---|---|---|---|---|---|---|---|---|---|
| LipiFlow ® | Provides thermal | S | One session | Required | Significant improvements in | Mild soreness, transient | Available in | $$$$ | $$-$$$ |
| | pulsation to unblock | | | | various clinical tests; SPEED | ocular discomfort that | certain eye | | |
| | MG and express | | | | improved and sustained for 3 | resolved without | care centers | | |
| | stagnant meibum | | | | years; OSDI returned to baseline | treatment | | | |
| | | | | | by 12 months post treatment | | | | |
| Intense Pulsed | Applies light energy | S | At least three | Not required | Significant improvements in | Pain, erythema, blistering; | Available in | $$$$ | $$ |
| Light (IPL) | to induce blood | | treatment | | various clinical tests after 3 | Sufficient eye protection | dermatology | | |
| | vessel ablation, | | sessions | | sessions of IPL | is required to avoid | or aesthetic | | |
| | reducing | | | | | damage to intraocular | clinics | | |
| | inflammation; heat | | | | | structures | | | |
| | from IPL also | | | | | | | | |
| | softens the meibum | | | | | | | | |
| NOV03 | Forms artificial lipid | S | Not | Not required | Clinically effective in reducing | None reported | Unclear | N/A | Unclear |
| | layer to reduce | | determined | | evaporation with significant | | | | |
| | evaporation from | | yet | | improvement in symptoms of | | | | |
| | ocular surface | | | | ocular dryness | | | | |
| iTear ® | Stimulate the | R | At least twice | Not required | Significant improvements in | Slight headache, sneezing, | Currently the | $$ | Unclear |
| | external nasal nerve | | per day | | Schirmer index and baseline | tickling sensation; | device is | | |
| | to activate natural | | | | OSDI after 30 days of treatment | lightheadedness, | available in a | | |
| | tear production | | | | | dizziness, nausea; | few countries | | |
| | | | | | | intermittent nose | and regions. | | |
| | | | | | | soreness; dry eye pain; | Treatment can | | |
| | | | | | | stye, papillae; itching and | be done in | | |
| | | | | | | sensitive eyes | home setting | | |

### 1. Diseases/Conditions to be Treated

In a preferred form, the formulation or composition described herein is applied to the eyelids of a subject to treat Meibomian gland dysfunction (MGD) in the subject.

MGD is a prevalent ocular condition characterized by obstruction and dysfunction of the meibomian glands, leading to insufficient lipid secretion and evaporative dry eye disease. MGD contributes to tear film instability, ocular surface inflammation, and discomfort, often resulting in symptoms such as dryness, irritation, redness, blurred vision, and foreign body sensation. The disclosed photothermal therapy effectively addresses these issues by restoring glandular function and improving lipid secretion, thereby enhancing tear film stability and overall ocular health.

In some forms, the formulation or composition described herein can be used in treating other ocular surface disorders associated with meibomian gland dysfunction and lipid layer abnormalities, such as blepharitis, evaporative dry eye disease, and contact lens discomfort due to compromised tear film stability. In some forms, the antimicrobial effects of ROS generation and controlled hyperthermia can be used in managing bacterial-related ocular infections, including anterior and posterior blepharitis. In some forms, the anti-inflammatory properties of the treatment is beneficial in reducing ocular surface inflammation associated with conditions like allergic conjunctivitis and rosacea-related ocular manifestations.

In some forms, the formulation or composition described herein may also be used for adjunct therapies for post-surgical recovery, particularly in managing inflammation and improving ocular surface healing following procedures such as laser eye surgery or cataract extraction.

### 2. Routes of Administration

In some forms, a method of treating an ocular disorder includes administering to the subject an effective amount of the disclosed compositions. For example, a pharmaceutical composition containing an effective amount of disclosed composition and one or more therapeutic agents can be applied to the eyelid of a subject in need thereof. Preferably, the composition is in a form suitable for direct application to the eyelid. In some forms, the composition is applied as a topical formulation.

In some forms, the radiation in a suitable wavelength is released from a portable or handheld probe, connected to a light, and a doctor or a patient need only align the probe to deliver the treatment or the subject can use the handheld device at home. In some forms, the light source is a fixed instrument in a clinic. In some forms, the light source is a transportable device e.g., a handheld LED infrared light. In some forms, the distance during treatment is approximately from about 2 cm to about 8 cm, or from about 3 cm to about 5 cm or 4 cm. In some forms, the duration of treatment is from about 1 min-20 min, 3 min-17 min, 5 min-15 min, 7 min-13 min, 8 min-12 min, 9 min-11 min, or 9-10 min for 1 day, 1-2 days, 1-3 days, 1-4 days or 1-5 days, 1-6 days, or 1-7 days.

In some forms, the source of radiation is adjustable to control at least one of radiation intensity, radiation wavelength, exposure duration, pulse frequency, or beam area.

### 3. Effective Amounts

The disclosed compositions can be administered to the eye therapeutically to achieve a therapeutic benefit, or prophylactically to achieve a prophylactic benefit, or to achieve both a therapeutic and prophylactic benefit. Therapeutic benefit means treating the underlying disorder including eradication or amelioration of one or more of the symptoms associated with the underlying disorder such that the patient reports an improvement in feeling or condition, notwithstanding that the patient can still be afflicted with the underlying disorder. For example, administration of a composition to a patient suffering from a condition provides therapeutic benefit when the patient reports a decrease in the severity or duration of the symptoms associated with the condition. Therapeutic benefit also includes halting or slowing the progression of the disease, regardless of whether improvement is realized by the patient.

In preferred embodiments, the disclosed compositions are retained at the site of administration (e.g., at the eyelid) for periods of time sufficient to yield a therapeutic effect.

It is to be understood that the disclosed method and formulations are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

In some forms, the photosensitive particles in the composition are present in a concentration of about 0.001 μg/mg-5.0 μg/mg, 0.005 μg/mg-2.5 μg/mg, 0.01 μg/mg-1.0 μg/mg, 0.015 μg/mg-0.95 μg/mg, 0.02 μg/mg-0.9 μg/mg, 0.03 μg/mg-0.8 μg/mg, 0.04 μg/mg-0.7 μg/mg, 0.05 μg/mg-0.6 μg/mg, 0.06 μg/mg-0.5 μg/mg, 0.07 μg/mg-0.4 μg/mg, 0.08 μg/mg-0.3 μg/mg, 0.09 μg/mg-0.2 μg/mg. In some forms, the photosensitive particles are present in a concentration of 0.1 μg/mg.

### EXAMPLES

### Example 1: a Light Sensitive Microscale-Dispersed Eye Ointment for the Treatment of Meibomian Gland Dysfunction

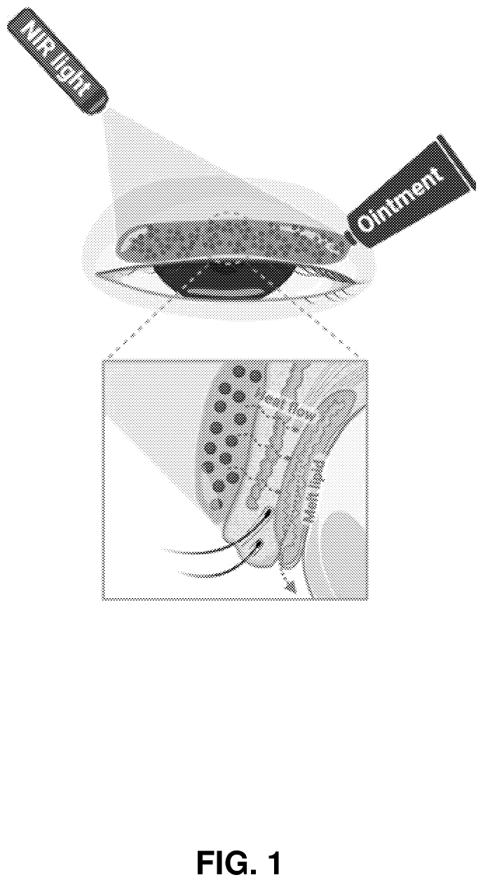

For MGD treatment, a photothermal therapy using a light sensitive microscale-dispersed eye ointment was made and its efficacy and safety tested on an MGD mouse model, as shown in FIG. 1.

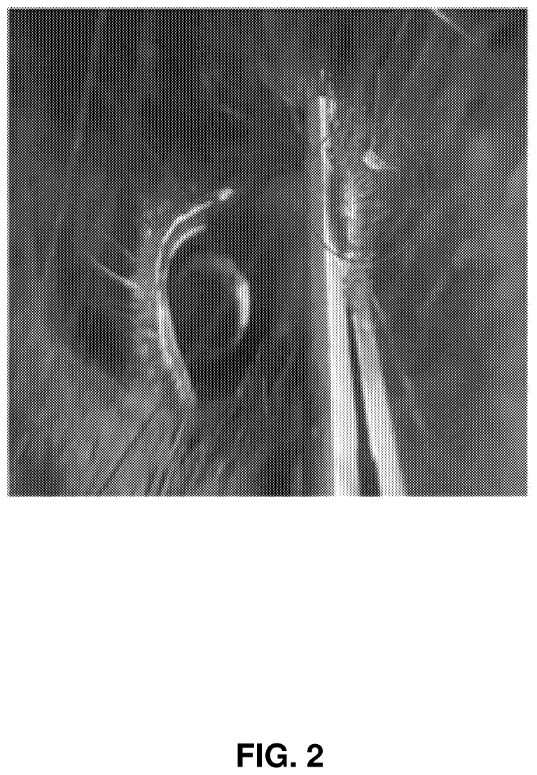

The MGD mouse model was established by feeding 10-week-old female ApoE-knockout mice with a high-fat diet for one month. When compressing the meibomian glands of the mice, pathological solidified meibum secretions (highlighted by red circles) were observed (FIG. 2), indicating successful establishment of the MGD mouse model. The light sensitive microscale-dispersed eye ointment consists of graphene and excipient (lanolin+Vaseline) with the graphene concentration of 0.1 μg/mg in the ointment. When treated with graphene ointment under irradiation of NIR at various currents, eyelid temperature of the mice showed a positive correlation with the current (FIG. 3). The treatment current was then fixed at 300 mA to reach an expected treatment temperature of around 41.3 °C for the Graphene-Near infrared light group (G-NIR). Treatment for the other experimental groups (Excipient-NIR group (E-NIR), Near-Infrared Light group (NIR)) were also carried out under this current for three days. The temperature should be maintained within an optimal range throughout the treatment to avoid either excessive heat that could damage the eyelid or insufficient heat that would reduce treatment efficacy. In the disclosed photothermal therapy system, the treatment temperature can be adjusted by regulating the current to reach the optimal range.

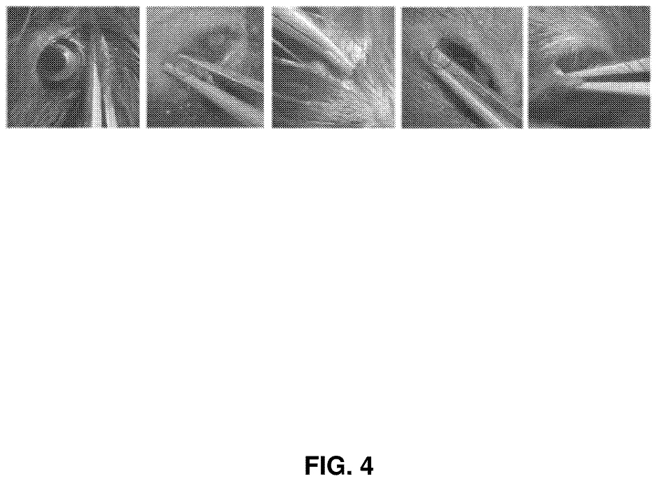

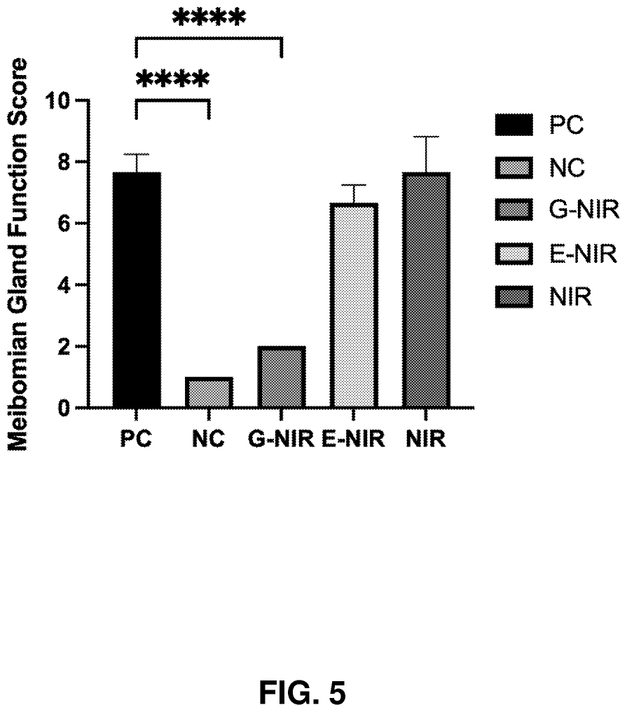

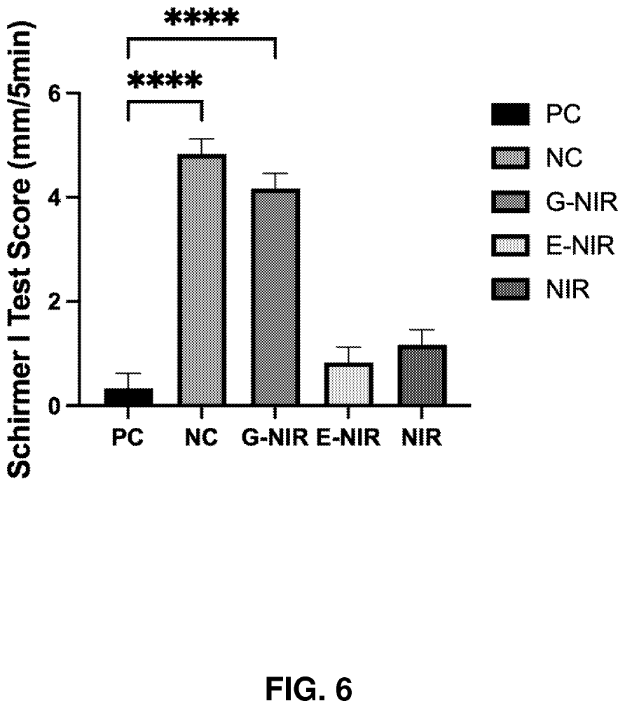

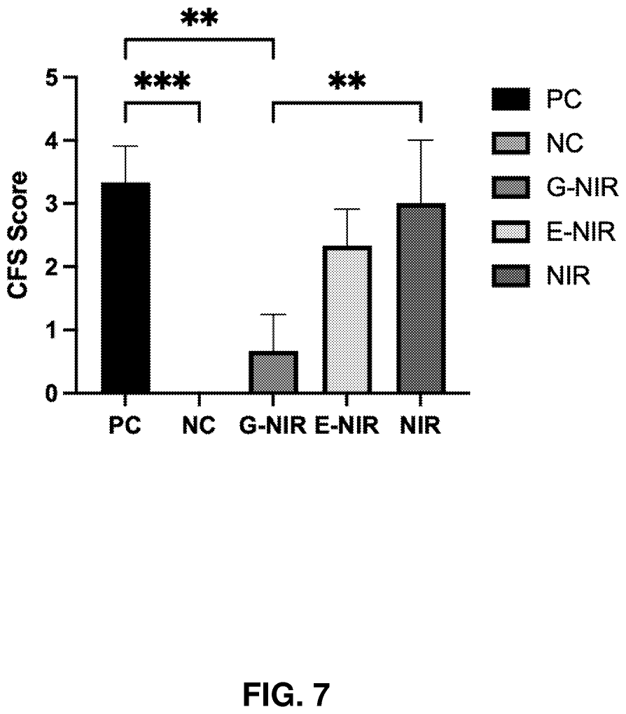

In clinical settings, meibomian gland function, tear secretion function, and MGD related corneal epithelium defect healing are three important examinations to assess meibomian gland status. Thus, these examinations were applied to evaluate the efficacy of the disclosed treatment. Meibomian gland function is evaluated through both qualitative and quantitative analysis of meibum. Upon meibomian gland compression, G-NIR and NC (normal control) groups exhibited clear and fluid meibum, while granular solidified meibum was observed in PC (positive control), E-NIR, and NIR groups (FIG. 4). The quantitative analysis of meibomian gland function is based on the Meibomian Gland Function Scoring of Chinese expert consensus on meibomian gland dysfunction: definition and classification (2023), in which a lower score represents better meibomian gland function. After three days of treatment, G-NIR group demonstrated a significant reduction in meibomian gland function score compared with PC, E-NIR, and NIR groups (FIG. 5). These results indicate effective improvements in meibomian gland function with the disclosed photothermal treatment, while NIR-only or NIR+ excipients treatments showed no obvious therapeutic effects. Furthermore, better performance of G-NIR group in Schirmer I test compared with PC, E-NIR, and NIR groups indicates improvements in tear secretion function (FIG. 6). Corneal epithelium defect healing was scored according to National Eye Institute Industry (NEI) Grading Scale for Corneal Fluorescein Staining, in which a lower score represents less corneal epithelium defect, referring to better meibomian gland status. The corneal fluorescein staining score of G-NIR group is significantly lower than those of PC, E-NIR, and NIR groups (FIG. 7). These results confirm the therapeutic efficacy of the disclosed treatment for MGD, aligning with the other results. In addition, no significant burns were observed on the eyelid skin or cornea, conjunctiva of mice after photothermal treatment, confirming its safety (FIG. 4).

All these results show that the disclosed light sensitive microscale-dispersed eye ointment can alleviate meibomian gland blockage and improve meibomian gland function. It has the potential to serve as an effective, economical, safe and user-friendly treatment for MGD in clinical settings.

It is understood that the disclosed method and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

Those skilled in the art will recognize or be able to ascertain using no more than routine experimentation, many equivalents to the specific embodiments of the method and compositions described herein. Such equivalents are intended to be encompassed by the following claims.

We claim:

1. An ocular composition comprising:
(i) one or more photosensitive particles capable of generating heat and/or reactive oxygen species (ROS) upon exposure to radiation; and
(ii) optionally, a pharmaceutically acceptable ocular carrier,
wherein the ocular composition is suitable for application to eyelids.

2. The ocular composition of claim 1, wherein the photosensitive particle is a microparticle, nanoparticle or a combination thereof.

3. The ocular composition of claim 1, wherein the photosensitive particle is made from a metal selected from the group consisting of derivative, gold, silver, and iron oxide.

4. The ocular composition of claim 1, wherein the photosensitive particle is carbon-based nanostructures made from a material selected from the group consisting of graphene, graphene derivatives, fullerenes or a semiconductor material.

5. The ocular composition of claim 1, wherein the photosensitive particle comprises one or more photoactive heterojunction material is selected from the group consisting of Graphene oxide/copper ferrite nanoparticles (Go/CFN), Silver-titanium oxide (Ag—TiO2), TiO2/AgVO3, Ag/TiO2/cellulose, Alginate/Au—TiO2, Lithium-titanate in the low-density polyethylene matrix (Li—TiO2/LDPE), Cu—TiO2, TiO2/—Fe2O3, TiO2 doped with Bi (Bi—TiO2 group) and TiO2 co-doped with urea and Bi (Urea, Bi—TiO2 group; U,Bi—TiO2), chitosan films containing melon/TiO2 (CTS/MTiO2), TiO2 nanoparticles and graphene sheets (TiO2/GSs), graphene oxide and cuprous oxide (rGO-Cu2O), and Zinc oxide-selenium (ZnO—Se), optionally, wherein the photoactive heterojunction material comprises Go/CFN and/or shows strong light absorbance at 808 nm.

6. The ocular composition of claim 1, wherein the photosensitive particle comprises graphene at a concentration of about 0.001 μg/mg-5.0 μg/mg, 0.005 μg/mg-2.5 μg/mg, 0.01 μg/mg-1.0 μg/mg, 0.015 μg/mg-0.95 μg/mg, 0.02 μg/mg-0.9 μg/mg, 0.03 μg/mg-0.8 μg/mg, 0.04 μg/mg-0.7 μg/mg, 0.05 μg/mg-0.6 μg/mg, 0.06 μg/mg-0.5 μg/mg, 0.07 μg/mg-0.4 μg/mg, 0.08 μg/mg-0.3 μg/mg, 0.09 μg/mg-0.2 μg/mg.

7. The ocular composition of claim 1, wherein the pharmaceutically acceptable ocular carrier is selected from the group consisting of liposomes, liposome-like nanoparticles, solid lipid nanoparticles, nanostructured lipid carriers, lipid-polymer hybrid nanoparticles, encapsulated lipid-polymer hybrid nanoparticles, and exosomes.

8. The ocular composition of claim 3, wherein the photosensitive particle is a microparticle.

9. The ocular composition of claim 1, wherein the carrier is selected from the group consisting of lanolin, petroleum jelly, mineral oil, wax, silicone oil, castor oil, coconut oil, synthetic esters, hydrogel or combination thereof.

10. The ocular composition of claim 1, further comprising one or more anti-inflammatory agents.

11. A formulation comprising the composition of claim 1, in a form selected from the group consisting of an ophthalmic gel, an ocular patch, a dissolvable or bioadhesive ocular film, an ocular wound bandage, an aerosolized or mist formulation, or a lipid-based ointment.

12. A method of treating an ocular condition in a subject in need thereof, comprising contacting an eye of the subject with the formulation of claim 10 and exposing the formulation to the radiation.

13. The method of claim 12, wherein the ocular condition is Meibomian gland dysfunction (MGD).

14. The method of claim 12, wherein a source of the radiation comprises one or more of a handheld device, a stationary light delivery system, a wearable device, a light-emitting diode (LED) array, a laser system, a broadband light source, a filtered light source, an intense pulsed light (IPL) device, or any combination thereof.

15. The method of claim 12, wherein the radiation has a wavelength of about 380 nm-1200 nm, 400 nm-1000 nm, 500 nm-900 nm, 550 nm-800 nm, 600 nm-750 nm, 1000 nm-1200 nm, 800 nm-1100 nm, or 900 nm-1000 nm.

16. The method of claim 15, wherein the radiation is near infrared (NIR) radiation.

17. The method of claim 12, wherein the radiation is exposed to the formulation on the eyelids for about 1 min-20 min, 3 min-17 min, 5 min-15 min, 7 min-13 min, 8 min-12 min, 9 min-11 min, or 9-10 min for 1 day, 1-2 days, 1-3 days, 1-4 days or 1-5 days, 1-6 days, or 1-7 days.

18. The method of claim 12, wherein exposing the formulation in the eyelid to the radiation generates heat and/or ROS at the eyelid.

19. The method of claim 12, wherein the treatment causes one or more of melting of solidified meibum, relief of Meibomian gland obstruction, reduction of ocular surface inflammation, improvement of meibum quality, or control of microbial growth.
